(19) United States
(12) Patent Application Publication
(10) Pub. No.: US 2026/0258339 A1
(43) Pub. Date: Sep. 3, 2026

(54) FIXED BED CELL CULTURE REACTOR VESSEL FOR SUBSTRATE ALIGNMENT AND SAMPLING

(71) Applicant: CORNING INCORPORATED, CORNING, NY (US)

(72) Inventors: Tristan Yale Brawn, Billerica, MA (US); Michael Francis Finnegan, Redwood City, CA (US); Jacob Dylan Freake, Cambridge, MA (US); Douglas Grosvenor Sabin, Marblehead, MA (US)

(21) Appl. No.: 18/877,771

(22) PCT Filed: Jun. 8, 2023
(86) PCT No.: PCT/US2023/024770

§ 371 Date: Dec. 20, 2024

Related U.S. Application Data
(60) Provisional application No. 63/354,401, filed on Jun. 22, 2022.

(51) Int. Cl. C12M 1/12; C12M 1/00

(52) U.S. Cl. CPC C12M 25/18; C12M 23/34; C12M 23/40; C12M 23/50; C12M 29/16

(57) ABSTRACT

A fixed bed bioreactor for culturing cells on a cell culture substrate is provided. The fixed bed bioreactor includes a cell culture vessel having a vessel body defining at least one interior reservoir, an inlet fluidly connected to the reservoir, and an outlet fluidly connected to the reservoir. The vessel body has a first end, a second end, and a longitudinal axis extending in a direction from the first end to the second. A guide rod is disposed in the interior reservoir and extends parallel to the longitudinal axis of the vessel body. The guide rod is configured to hold in place the cell culture substrate in the interior reservoir.

### CROSS-REFERENCE TO RELATED APPLICATIONS

This application claims the benefit of priority under 35 U.S.C. § 119 of U.S. Provisional Application Ser. No. 63/354,401 filed on Jun. 22, 2022, the content of which is relied upon and incorporated herein by reference in its entirety.

### FIELD OF THE DISCLOSURE

This disclosure generally relates to cell culture bioreactors and substrates for culturing cells. In particular, the present disclosure relates to cell culturing substrates and bioreactors incorporating such substrates that enable defined packing and/or sampling of the substrate.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of packed bed systems disclosed in a prior art is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

Some existing bioreactor solutions use small strips of cell substrate material consisting of randomly oriented fibers in a non-woven arrangement. These strips are packed into a vessel to create a packed bed. However, as with similar solutions on the market, there are drawbacks to this type of packed-bed substrate. Specifically, non-uniform packing of the substrate strips creates visible channels within the packed bed, leading to preferential and non-uniform media flow and nutrient distribution through the packed bed. Studies of such systems have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the packed bed may improve dispersion, but would have other drawbacks (i.e., “necessary agitation for better dispersion during inoculation and transfection would induce increased shear stress, in turn leading to reduced cell viability.” Id.). Another study noted that the uneven distribution of cells makes monitoring of the cell population using biomass sensors difficult (“ . . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between one packed bed and another in bioreactors like those discussed above, it can be difficult for customers to predict cell culture performance, since the substrate varies between cultures. Furthermore, the randomly packed substrate, which themselves have random structures, makes efficiently harvesting cells very difficult or impossible, as it is believed that cells are entrapped by the packed bed.

While manufacturing of viral vectors for early-phase clinical trials is possible with existing platforms, there is a need for a platform that can produce high-quality product in greater numbers in order to reach late-stage commercial manufacturing scale.

In addition, it is desirable to be able to monitor bioreactors used to culture cells or make AAV or to create a seed train to facilitate cell expansion for biochemical production. When using adherent cell reactors, samples of the growth media do not contain cells, or at least not to an extent that is useful for monitoring the state of the culture on the adherent substrate.

There is a need for cell culture matrices, systems, and methods that enable culturing of cells in a high-density format, with uniform cell distribution, and easily attainable and increased harvesting yields, while also enabling users to monitor the state of the cell culture process by examining the cells on the substrate during and/or after the cell culture process, including monitoring the substrate aseptically.

### SUMMARY

According to embodiments of this disclosure, a cell culture substrate is disclosed that allows for sampling all or a portion of the substrate to monitor the status or health of the cell culture. Embodiments include a multilayered fixed bed cell culture matrix with one or more layers specifically designed to enable this sampling. Embodiments also include a fixed bed bioreactor with such cell culture substrates and/or matrices, including bioreactor vessels that enable arrangement cell culture substrates in a defined arrangement and orientation.

To be able to estimate the number of cells and their distribution and health in an adherent cell bioreactor bed, one method uses the removal of a portion of the substrate in the midst of the cell culture or cell expansion process. By sampling during the process, information about the cell culture run can be used to assess the quality and performance of the process. Cell count can be estimated from the sample and growth can be monitored by sampling at different times. This information can be used to develop and optimize performance of specific biological processes such as seed train and viral vector production. In production, runs that are contaminated or out of specification, can be terminated to reduce the cost of running the process to its end without a satisfactory result. Growth media and lost production time represent significant cost for typical biological processes. Embodiments of this disclosure allow all or portions of the fixed bed cell culture substrate to be removed from the housing to give users access to the bed without destroying the bed or bioreactor vessel. This allows any portion or select portions of the fixed bed to be assessed. The bed can also be accessed after the cell culture process or harvesting of the desired component is completed for a “post-mortem” analysis of the cell culture.

A fixed bed bioreactor for culturing cells on a cell culture substrate is provided. The fixed bed bioreactor includes a cell culture vessel having a vessel body defining at least one interior reservoir, an inlet fluidly connected to the reservoir, and an outlet fluidly connected to the reservoir. The vessel body has a first end, a second end, and a longitudinal axis extending in a direction from the first end to the second. A guide rod is disposed in the interior reservoir and extends parallel to the longitudinal axis of the vessel body. The guide rod is configured to hold in place the cell culture substrate in the interior reservoir.

### BRIEF DESCRIPTION OF THE DRAWINGS

FIG. 1A shows a perspective view of a three-dimensional model of a cell culture substrate, according to embodiments of this disclosure.

FIG. 1B is a two-dimensional plan view of the substrate of FIG. 1A.

FIG. 1C is a cross-section along line A-A of the substrate in FIG. 1B.

FIG. 2A shows a perspective view of a multilayer cell culture substrate, according to embodiments.

FIG. 2B shows a plan view of a multilayer cell culture substrate, according to embodiments.

FIG. 3 shows a cross-section view along line B-B of the multilayer cell culture substrate of FIG. 2B, according to embodiments.

FIG. 4 shows a cross-section view along line C-C of the multilayer cell culture substrate of FIG. 3, according to embodiments.

FIG. 5 shows a schematic view of a cell culture system, according to embodiments.

FIG. 6A shows a plan view of a cell culture substrate sample layer with a separation boundary defining a substrate sample portion, according to one or more embodiments.

FIG. 6B shows a plan view of the cell culture substrate sample layer and sample portion of FIG. 6B after the sample portion is separated from the remainder of the substrate sample layer, according to one or more embodiments.

FIG. 7A shows a plan view ell culture substrate sample layer with a plurality of substrate sample portions having tapered ends, according to one or more embodiments.

FIG. 7B shows an individual substrate sample portion from FIG. 8B.

FIG. 7C shows the individual substrate sample portion of FIG. 8B and a port through which the substrate sample portion can be extracted from a fixed bed cell culture substrate.

FIG. 8 is a plan view of a substrate sample layer with tethered sample portions, according to some embodiments.

FIG. 9A is a photograph of a substrate sample layer of woven PET mesh, according to some embodiments.

FIG. 9B is a photograph of crystal violet stained sample portions of woven PET mesh prior to harvesting the adherent cells, according to some embodiments.

FIG. 9C is a photograph of crystal violet stained sample portions of woven PET mesh substrate after harvesting the adherent cells, according to some embodiments.

FIG. 10 is an exploded view of a fixed bed reactor for cell culture, according to embodiments.

FIG. 11 shows a cell culture bed and guide rod with keyholes features, according to embodiments.

FIG. 12 is a diagram of a fixed bed reactor vessel have a removable substrate for sampling, according to embodiments.

FIG. 13 is a close-up view of the section 1213 from FIG. 12.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include cell culture substrates, as well as cell culture bioreactors incorporating such a substrate, that enabling sampling of the substrate or a portion of the substrate for monitoring cell culture.

In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

According to some embodiments, a method of cell culturing is also provided using bioreactors with the matrix for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

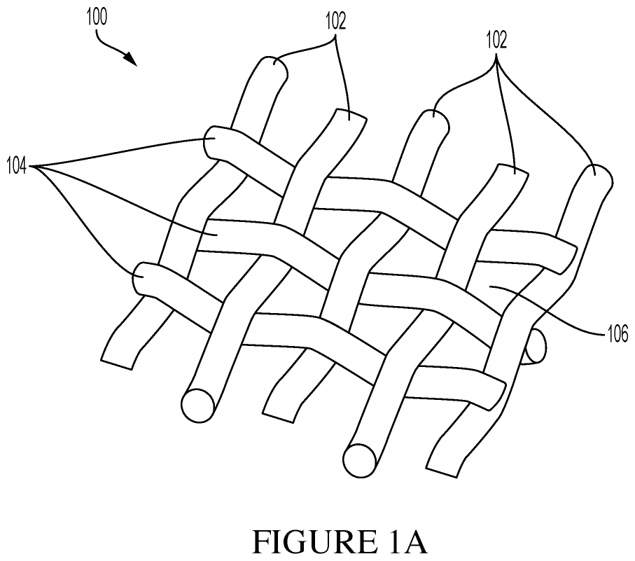

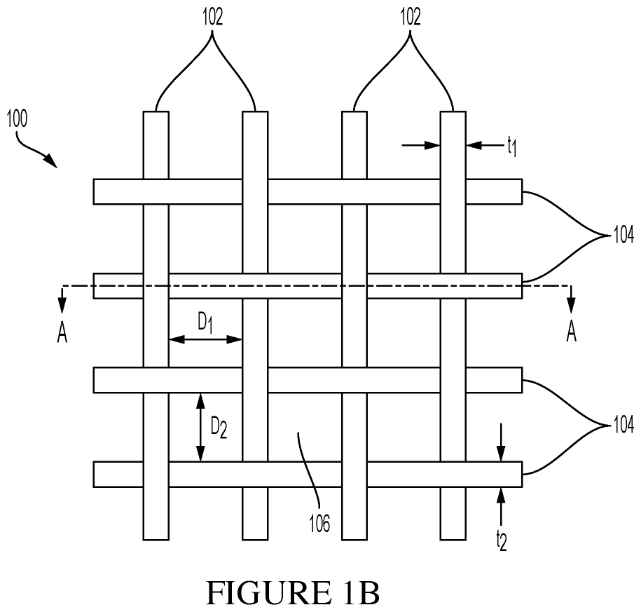

FIGS. 1A and 1B show a three-dimensional (3D) perspective view and a two-dimensional (2D) plan view, respectively, of a cell culture substrate 100, according to an example of one or more embodiments of this disclosure. The cell culture substrate 100 is a woven mesh layer made of a first plurality of fibers 102 running in a first direction and a second plurality of fibers 104 running in a second direction. The woven fibers of the substrate 100 form a plurality of openings 106, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 1C, a thickness T of the woven mesh 100 may be thicker than the thickness of a single fiber (e.g., t₁). As used herein, the thickness T is the maximum thickness between a first side 108 and a second side 110 of the woven mesh. Without wishing to be bound by theory, it is believed that the three-dimensional structure of the substrate 100 is advantageous as it provides a large surface area for culturing adherent cells, and the structural rigidity of the mesh can provide a consistent and predictable cell culture matrix structure that enables uniform fluid flow.

In FIG. 1B, the openings 106 have a diameter D₁, defined as a distance between opposite fibers 102, and a diameter D₂, defined as a distance between opposite fibers 104. D₁ and D₂ can be equal or unequal, depending on the weave geometry. Where D₁ and D₂ are unequal, the larger can be referred to as the major diameter, and the smaller as the minor diameter. In some embodiments, the diameter of an opening may refer to the widest part of the opening. Unless otherwise specified, the opening diameter, as used herein, will refer to a distance between parallel fibers on opposite sides of an opening.

A given fiber of the plurality of fibers 102 has a thickness t₁, and a given fiber of the plurality of fibers 104 has a thickness t₂. In the case of fibers of round cross-section, as shown in FIG. 1A, or other three-dimensional cross-sections, the thicknesses t₁ and t₂ are the maximum diameters or thicknesses of the fiber cross-section. According to some embodiments, the plurality of fibers 102 all have the same thickness t₁, and the plurality of fiber 104 all have the same thickness t₂. In addition, t₁ and t₂ may be equal. However, in one or more embodiments, t₁ and t₂ are not equal such as when the plurality of fibers 102 are different from the plurality of fiber 104. In addition, each of the plurality of fibers 102 and plurality of fibers 104 may contain fibers of two or more different thicknesses (e.g., t₁ₐ, t_{1b}, etc., and t₂ₐ, t_{2b}, etc.). According to embodiments, the thicknesses t₁ and t₂ are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 1A-1C, the 2D surface area of the fibers available for cell attachment and proliferation exceeds the surface area for attachment on an equivalent planar 2D surface.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm x 100 μm to about 1000 μm×1000 μm. In some embodiments, the opening may have a diameter o about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix is comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm² . The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm² . These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

By using a structurally defined culture matrix of sufficient rigidity, high-flow-resistance uniformity across the matrix or packed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

As used herein, “structurally defined” means that the structure of the substrate follows a predetermined design and is not random. The structurally defined substrate can thus be a woven design, 3D printed, molded, or formed by some other technique known in the art that allows the structure to follow a predetermined planned structure.

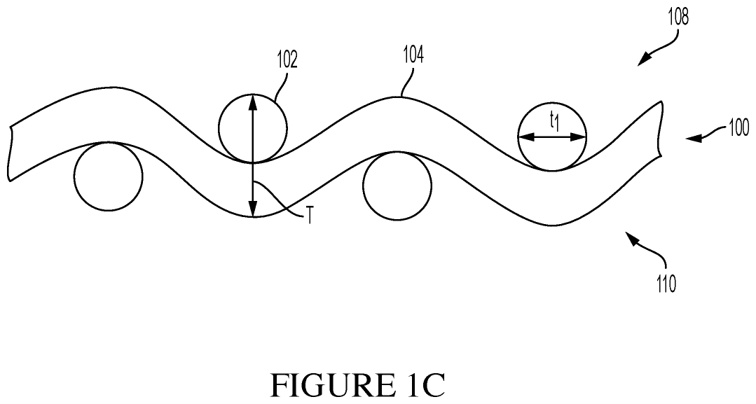

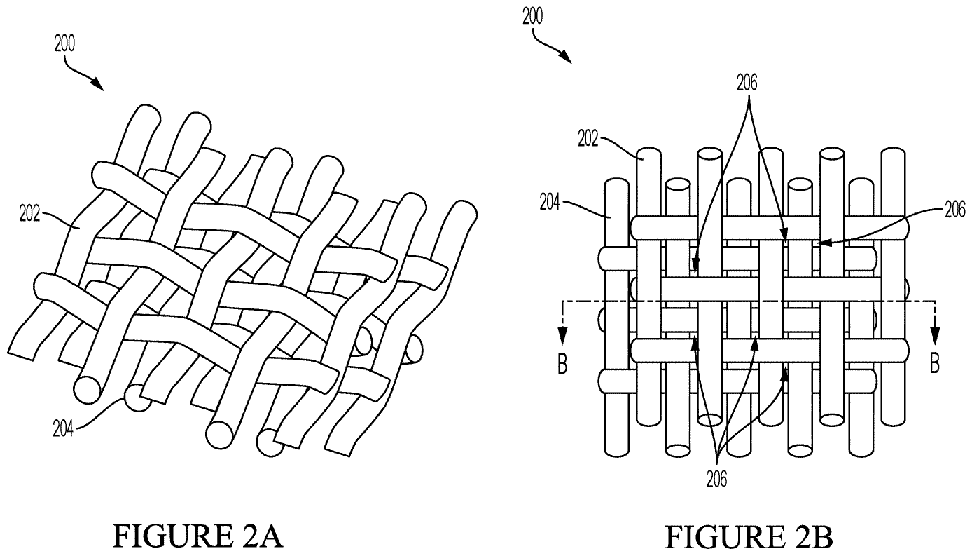

FIG. 2A shows an embodiment of the matrix with a multilayer substrate 200, and FIG. 2B is a plan view of the same multilayer substrate 200. The multilayer substrate 200 includes a first mesh substrate layer 202 and a second mesh substrate layer 204. Despite the overlapping of the first and second substrate layers 202 and 204, the mesh geometries (e.g., ratio of opening diameters to fiber diameters) is such that the openings of the first and second substrate layers 202 and 204 overlap and provide paths for fluid to flow through the total thickness of the multilayer substrate 200, as shown by the filament-free openings 206 in FIG. 2B.

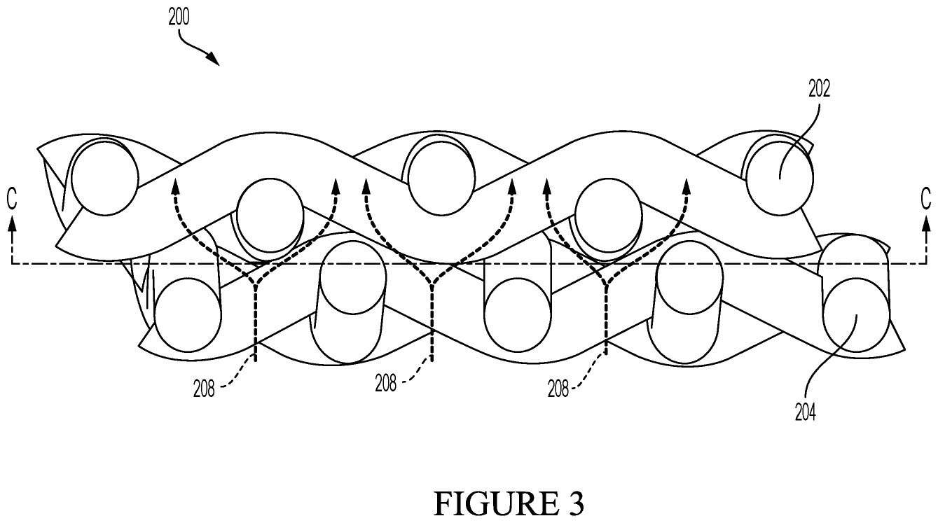

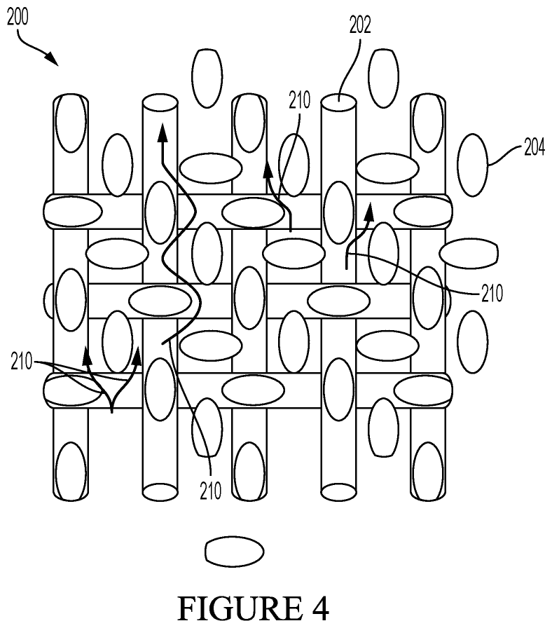

FIG. 3 shows a cross section view of the multilayer substrate 200 at line B-B in FIG. 2B. The arrows 208 show the possible fluid flow paths through openings in the second substrate layer 204 and then around filaments in the first substrate layer 202. The geometry of the mesh substrate layers is designed to allow efficient and uniform flow through one or multiple substrate layers. In addition, the structure of the matrix 200 can accommodate fluid flow through the matrix in multiple orientations. For example, as shown in FIG. 3, the direction of bulk fluid flow (as shown by arrows 208) is perpendicular to the major side surfaces of the first and second substrate layers 202 and 204. However, the matrix can also be oriented with respect to the flow such that the sides of the substrate layers are parallel to the bulk flow direction. FIG. 4 shows a cross section view of the multilayer substrate 200 along line C-C in FIG. 3, and the structure of matrix 200 allows for fluid flow (arrows 210) through fluid pathways in the multilayer substrate 200. In addition to fluid flow being perpendicular or parallel to the first and second sides of the mesh layers, the matrix can be arranged with multiple pieces of substrate at intermediate angles, or even in random arrangements with respect to fluid flow. This flexibility in orientation is enabled by the essentially isotropic flow behavior of the woven substrate. In contrast, substrates for adherent cells in existing bioreactors do not exhibit this behavior and instead their packed beds tend to create preferential flow channels and have substrate materials with anisotropic permeability. The flexibility of the matrix of the current disclosure allows for its use in various applications and bioreactor or container designs while enabling better and more uniform permeability throughout the bioreactor vessel.

As discussed herein, the cell culture substrate can be used within a bioreactor vessel, according to one or more embodiments. For example, the substrate can be used in a packed bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

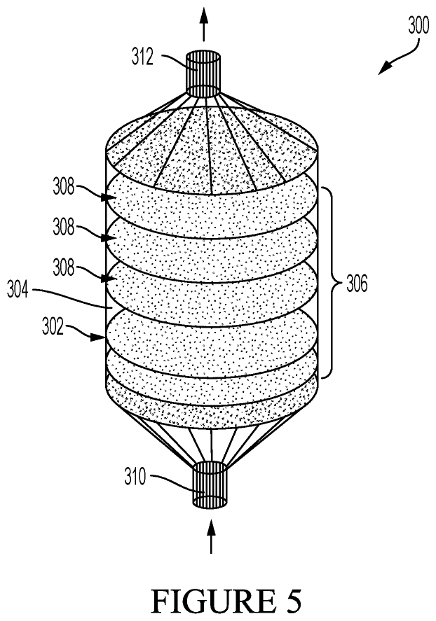

A cell culture system is provided, according to one or more embodiments, in which the cell culture matrix is used within a culture chamber of a bioreactor vessel. FIG. 5 shows an example of a cell culture system 300 that includes a bioreactor vessel 302 having a cell culture chamber 304 in the interior of the bioreactor vessel 302. Within the cell culture chamber 304 is a cell culture matrix 306 that is made from a stack of substrate layers 308. The substrate layers 308 are stacked with the first or second side of a substrate layer facing a first or second side of an adjacent substrate layer. The bioreactor vessel 300 has an inlet 310 at one end for the input of media, cells, and/or nutrients into the culture chamber 304, and an outlet 312 at the opposite end for removing media, cells, or cell products from the culture chamber 304. By allowing stacking of substrate layers in this way, the system can be easily scaled up without negative impacts on cell attachment and proliferation, due to the defined structure and efficient fluid flow through the stacked substrates. While the vessel 300 may generally be described as having an inlet 310 and an outlet 312, some embodiments may use one or both of the inlet 310 and outlet 312 for flowing media, cells, or other contents both into and out of the culture chamber 304. For example, inlet 310 may be used for flowing media or cells into the culture chamber 304 during cell seeding, perfusion, or culturing phases, but may also be used for removing one or more of media, cells, or cell products through the inlet 310 in a harvesting phase. Thus, the terms “inlet” and “outlet” are not intended to restrict the function of those openings.

In one or more embodiments, flow resistance and volumetric density of the packed bed can be controlled by interleaving substrate layers of different geometries. In particular, mesh size and geometry (e.g., fiber diameter, opening diameter, and/or opening geometry) define the fluid flow resistance in packed bed format. By interlaying meshes of different sizes and geometries, flow resistance can be controlled or varied in one or more specific portions of the bioreactor. This will enable better uniformity of liquid perfusion in the packed bed. For example, 10 layers of Mesh A (Table 1) followed by 10 layers of Mesh B (Table 1) and followed by 10 layers of Mesh C (Table 1) can be stacked to achieve a desired packed bed characteristic. As another example, the packed bed may start with 10 layers of Mesh B, followed by 50 layers of Mesh C, followed by 10 layers of Mesh B. Such repetition pattern may continue until the full bioreactor is packed with mesh. These are examples only, and used for illustrative purposes without intending to be limiting on the possible combinations. Indeed, various combinations of meshes of different sizes are possible to obtain different profiles of volumetric density of cells growth surface and flow resistance. For example, a packed bed column with zones of varying volumetric cells densities (e.g., a series of zones creating a pattern of low/high/low/high, etc. densities) can be assembled by interleaving meshes of different sizes.

In FIG. 5, the bulk flow direction is in a direction from the inlet 310 to the outlet 312, and, in this example, the first and second major sides of the substrate layers 308 are perpendicular to the bulk flow direction.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. As discussed above, the substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In one or more embodiments, the cell culture matrix is secured within the culture chamber by a fixing mechanism. The fixing mechanism may secure a portion of the cell culture matrix to a wall of the culture chamber that surrounds the matrix, or to a chamber wall at one end of the culture chamber. In some embodiments, the fixing mechanism adheres a portion of the cell culture matrix to a member running through the culture chamber, such as member running parallel to the longitudinal axis of the culture chamber, or to a member running perpendicular to the longitudinal axis. However, in one or more other embodiments, the cell culture matrix may be contained within the culture chamber without being fixedly attached to the wall of the chamber or bioreactor vessel. For example, the matrix may be contained by the boundaries of the culture chamber or other structural members within the chamber such that the matrix is held within a predetermined area of the bioreactor vessel without the matrix being fixedly secured to those boundaries or structural members.

By using a cell culture matrix according to embodiments of this disclosure, such as a matrix including a woven or mesh substrate, the roller bottle vessel is provided with an increased surface area available for adherent cells to attach, proliferate, and function. In particular, using a substrate of a woven mesh of monofilament polymer material within the roller bottle, the surface area may increase by of about 2.4 to about 4.8 times, or to about 10 times that of a standard roller bottle. As discussed herein, each monofilament strand of the mesh substrate is capable of presenting itself as 2D surface for adherent cells to attach. In addition, multiple layers of mesh can we arranged in roller bottle, resulting in increases of total available surface area ranging from about 2 to 20 times that of a standard roller bottle. Thus, existing roller bottle facilities and processing, including cell seeding, media exchange, and cell harvesting, can be modified by the addition of the improved cell culture matrix disclosed herein, with minimal impact on existing operation infrastructure and processing steps.

The bioreactor vessel optionally includes one or more outlets capable of being attached to inlet and/or outlet means. Through the one or more outlets, liquid, media, or cells can be supplied to or removed from the chamber. A single port in the vessel may act as both the inlet and outlet, or multiple ports may be provided for dedicated inlets and outlets.

The packed bed cell culture matrix of one or more embodiments can consist of the woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizonal or vertical axis.

This disclosure describes substrates and methods to cut and perforate layers of cell culture substrate, including polymer mesh substrates, to create a detachable sample piece. The disclosure also describes methods and apparatus to aseptically remove the sample from a bioreactor. By sampling during the cell culture process, information about the run can be used to assess the quality and performance of the culture process. Cell count can be estimated from the sample and growth can be monitored by sampling at different times or at different places within the bioreactor. This information can be used to develop and optimize parameters for specific biological processes such as seed train and viral vector production. In production, processes that are contaminated or out of specification, can be terminated to reduce the cost of running the process to its end without a satisfactory result. Growth media and lost production time represent significant cost for typical biological processes.

In embodiments herein, substrate sample portions are separable from a remainder of the cell culture substrate with a low force to allow sampling to be accomplished ideally by hand and without disturbing the main mesh body during the sampling process. For example, a relatively low (e.g., applied by hand) force can cause the sample portion to separate from the remainder of the substrate via the tension between the sample portion to which the force is applied and the remainder of the substrate. To keep the removal force low, the separation boundary can be applied between the sample portion and the remainder of the substrate. This separation boundary can be formed, for example, by scoring, perforation, laser cutting, or other cutting means such as die cutting, and can be used to create a layer of substrate that includes separable pieces of the substrate that can be removed from the fixed bed.

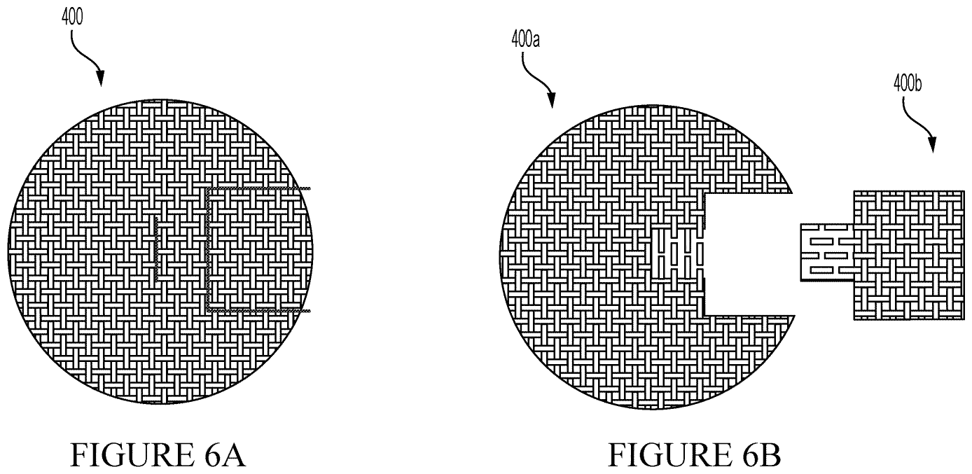

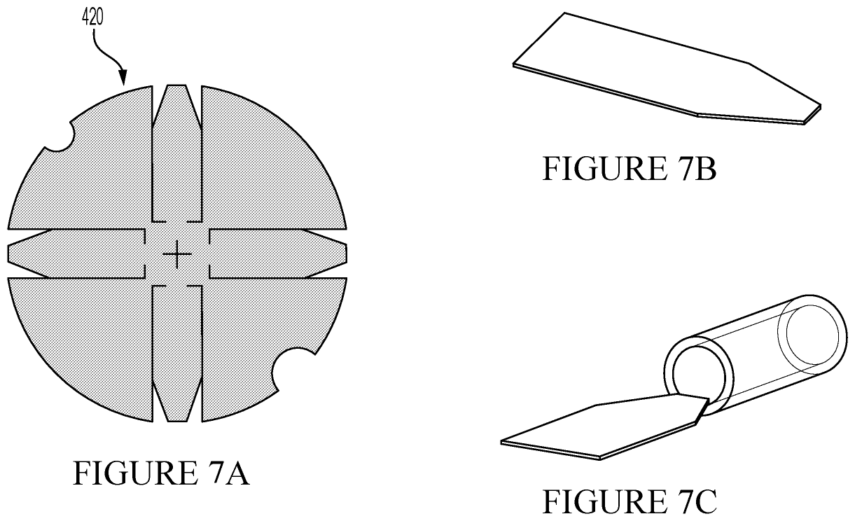

Some embodiments use woven polymer mesh substrates that have woven fibers defining an ordered array of pores or openings. Because each of the fibers in the mesh is very strong, it is desirable to have no fibers that run between the detachable sample and the main body of the mesh to facilitate the sample being removed with a low force. It is also desirable to have the mesh layer be robust when handled during the manufacturing and assembly process used to create a mesh stack bioreactor bed. To accomplish this, some fibers can be cut in such a way to leave a woven portion of the mesh that connects the sample to the main mesh body as shown in FIG. 7A. Due to the relative stiffness of the fibers, which may be formed from a variety of polymers disclosed herein (including PET), the interwoven fibers may remain attached even though individual fibers are severed to create the separation boundary of the sample portion. The lines in FIG. 6A show the separation boundary. FIG. 6B shows the sample portion after it has detached from the remainder of the cell culture substrate.

Mesh layers with sample pieces cut can be removed from bioreactors by opening the bioreactor housing and pulling them off the bed with a sterile tool or they can be removed from the reactor by using an aseptic sampling port.

A multiplicity of sampling pieces can come from a single layer. In embodiments using woven substrates, the direction of the warp and the weave as it interacts with the cutting pattern for most cutting patterns is considered to maintain the structural integrity and enable easy removal. Some cutting patterns created are less sensitive to the orientation of the mesh fibers and these patterns are advantages to use in manufacturing because the mesh orientation does not need to be precisely controlled.

FIGS. 7A-7C show another embodiment in which the sampling layer contains multiple sampling portions. The shape of the sampling portions includes a rectangular end on the interior side of the sampling portion within the periphery of the sampling layer, and a tapered end on the exterior end at the periphery of the sampling layer. The tapered end allows for easy removal of the sampling portion through a port in the sidewall of the bioreactor. The substrate material is such that the size of the port in the sidewall can be at or just larger than the side of the narrow end, and the wider portion of the sampling portion can slightly fold or curve as it is pulled through the opening in the sidewall. FIG. 7B shows a close up view of an individual sampling portion after being detached, and FIG. 7C shows an example of the relative size between the sidewall port in the bioreactor and the sample portion, although the relative sizes can vary in various embodiments.

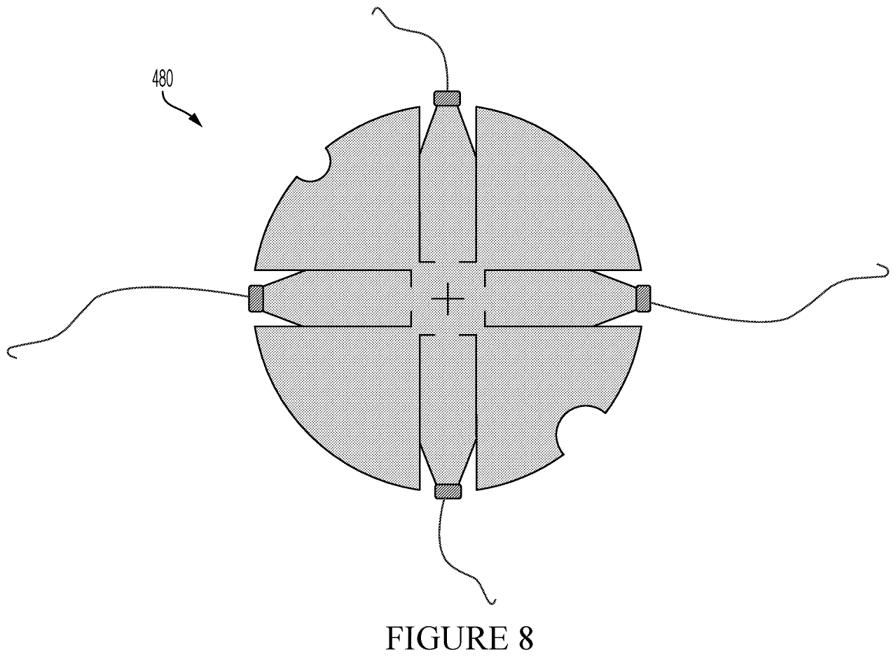

FIG. 8 shows an embodiment where sample layers of substrate have tethers molded to the sample portions, such that the tethers can be pulled to remove the sample portions. Embodiments include a method of assembling a bioreactor in which layers of substrate are added to the bioreactor housing until the sampling port elevation is reached. At this point a sampling layer is inserted into the bioreactor vessel and the tethers are pulled through the ports. Aseptic containers on the exterior of the ports can be used to allow aseptic sampling.

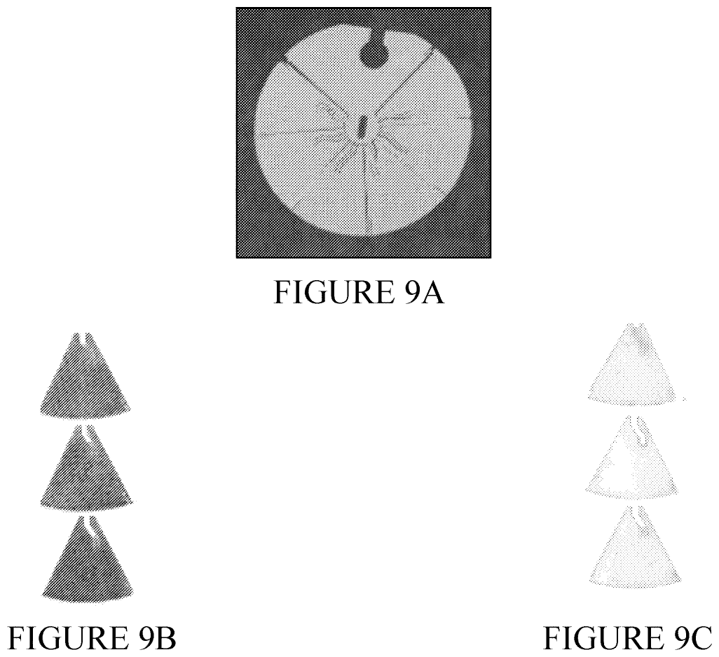

FIG. 9A shows a sample layer having six pie-shaped sampling portions. The number and shape of the sampling portions can vary. In this case, the separation boundary is laser cut through the fibers of the woven mesh substrate. The sample layer also includes an alignment feature on the left side of the layer, which can be useful for keeping the sample layer in a predetermined position so that the sampling portions are in a predetermined position for easy sampling. The alignment feature can be designed to mate with a corresponding feature on the interior of the vessel sidewall. FIG. 9B shows three pie-shaped sample portions that have been stained to show the presence of adherent cells on the substrate. FIG. 9C shows three pie-shaped sample portions that have been sampled after a harvesting procedure to harvest the cells from the substrate. Comparing FIGS. 9B and 9C, shows the effectiveness of the harvesting procedure in this example.

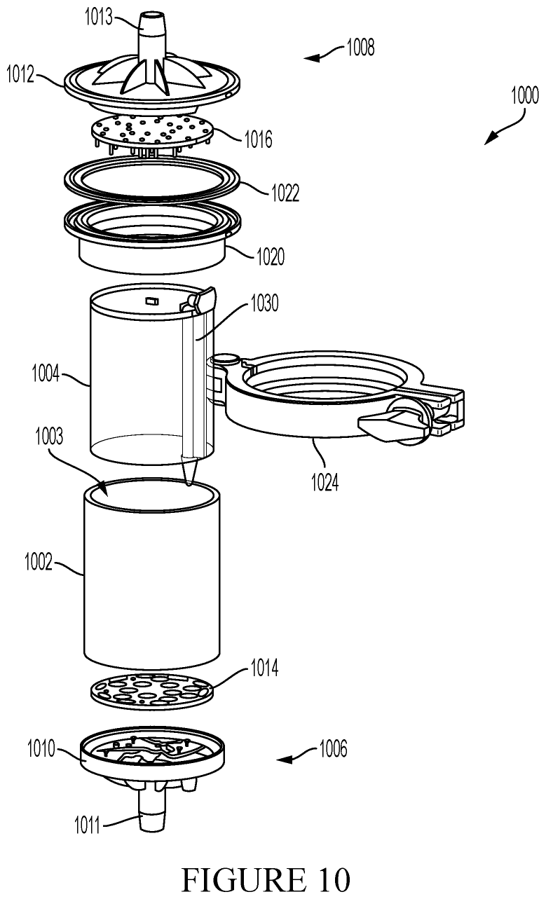

FIG. 10 shows an exploded view of a fixed bed reactor vessel 1000, according to embodiments of this disclosure. The vessel 1000 includes a body 1002, shown in FIG. 10 as a cylindrical vessel wall, that encloses a cell culture space 1003 for containing a cell culture substrate 1004. The cell culture substrate 1004 can include one or more porous materials, such as those disclosed herein, in a stacked or rolled arrangement. Embodiments of this disclosure include fixed bed reactor vessels 1000 can take provide for easy sampling of the cell culture substrate 1004 and/or for controlled positioning of the cell culture substrate 1004 within the cell culture space 1003. For example, as explained below, the vessel 1000 may help maintain a position of the cell culture substrate 10004, such that the cell culture substrate 1004 does not shift during operation, or such that layers of the cell culture substrate 1004 are disposed within the cell culture space 1003 at a predetermined orientation with respect to each other.

The vessel 1000 has a first end 1006 and a second end 1008 on either end of the body 1002 through which media, cells, and/or cell byproducts can be introduced into and/or removed from the cell culture space 1003. The first and second ends 1006, 1008 can be sealed to retain the sanitary conditions of the cell culture space by bottom end cap 1010 and top end cap 1012, respectively. Each of the bottom and top end caps 1010, 1012 is provided with one ore more fluid inlets and/or outlets 1011, 1013 for fluid flow through the bioreactor vessel 1000 during operation. An inlet distributor plate 1014 can be used adjacent to the bottom end cap 1010 for evenly distributing fluid across a cross-section of the cell culture space 1003. Similarly, an outlet distributor plate 1016 or collection plate can be used adjacent to the top end cap 1012 to help gather fluid from across the cell culture space 1003 and direct it towards an outlet in the top end cap 1012. The use of the inlet and outlet distributor plates 1014 and 1016 helps improve flow uniformity through the cell culture space 1003 by promoting the even and uniform flux of fluid across the cross-sectional area of the cell culture space 1003 at the bottom and top of the cell culture substrate 1004. Although the inlet and outlet distributor plates 1014 and 1016 are shown in FIG. 10 as discrete components from the bottom and top end caps 1010 and 1012, it is contemplated that the distributor plates 1014, 1016 can be integral with the end caps 1010, 1012.

The reactor vessel 1000 can be enclosed using a variety of closure mechanisms, including one or more of welded seals, adhesives, clamps, and gaskets. For example, FIG. 10 shows a sanitary flange 1020 at the top of the cell culture space 1003. The sanitary flange 1020 mates with a sanitary seal 1022 disposed between the sanitary flange 1020 and the top end cap 1012. A sanitary clamp 1024 can securely mate the top end cap 1012 to the sanitary flange 1020. A benefit of using this arrangement is that the sanitary clamp 1024 can easily be removed to provide access to the cell culture space 1003 (e.g., for sampling of the cell culture during use).

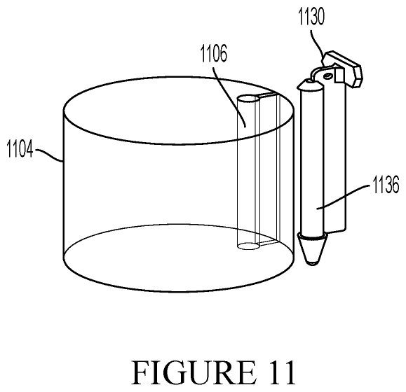

The reactor vessel 1000 also includes a guide rod 1030. The guide rod 1030 extends through the cell culture space 1003 and is used to align the cell culture substrate 1004 within the cell culture space 1003 and/or to align layers of the cell culture substrate 1004 with each other. For example, as shown in FIG. 11, a cell culture substrate fixed bed 1104 can be keyed to the guide rod 1130 via a substrate alignment feature 1106. The substrate alignment feature 1106 is designed to engage with a rod alignment feature 1136 in such a way that the fixed bed and/or individual substrate layers in the fixed bed remain in a desired location and/or orientation. In embodiments where the fixed bed includes multiple layers of stacked substrate material, for example, it may be desired to have each individual layer at a specific orientation to other layers. For example, in the case of substate layers having a defined structure of rigid fibers arranged in a predetermined direction, it can be desirable to vary the orientation of the fibers in layers within the fixed bed. In embodiments, this may include rotating layers a predetermined angle about a central longitudinal axis of the cell culture space 1003. If adjacent layers are rotated with respect to each other, this can allow for controlling of the packing density of the layers by controlling the amount of nesting that occurs between adjacent layers. This also effects the porosity and/or pore geometry of the packed bed, which effects fluid flow through the fixed bed. For example, in some embodiments, each layer may be rotated about 45° with respect to one or both adjacent layers in the stack, for improve fluid flow and cell culture performance. Taking this as an example, the substrate alignment feature 1106 may be a cutout in each layer of the fixed bed, where the position of the substrate alignment feature 1106 is rotational displaced around the layers to result in the relative rotation of the layers when the substrate alignment features 1106 of multiple layers are aligned. The corresponding rod alignment feature 1136 then keeps the substate alignment features 1106 aligned, which in turn keeps the individual layers in their desired orientation.

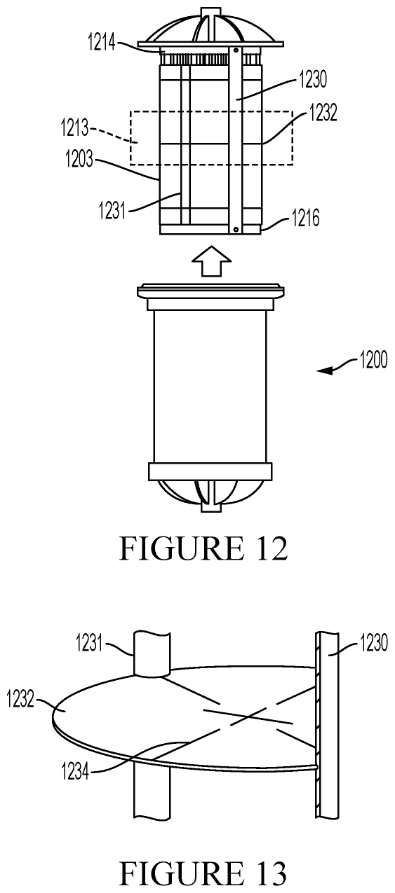

As shown in FIG. 12, a guide rod 1230 can be attached to structures (such as the distributor plates 1214, 1216) on either end of the cell culture substrate 1203 so that the guide rod 1230 confines the cell culture substrate 1203 therein and allowing the entire fixed bed to be removed from a remainder of the fixed bed reactor containing the vessel wall 1200. This allows for easy removal and sampling of the fixed bed without disrupting a remainder of the fixed bed, or disturbing the alignment of the substate layers, for example. FIG. 13 shows an example of embodiments in which a sample substrate layer 1232 contained within the fixed bed has one or more perforations 1234, allowing for easy removal of a portion of the substrate 1232. It is contemplated that an additional guide rod 1231 or other structure member may be provided for reinforce the structural integrity of the removable substate core. Embodiments are not limited to the specific arrangement of guide rods, distributor plates, and/or sample mesh shown in FIGS. 12 and 13, but rather illustrate an example of various embodiments to accomplish sampling from the reactor vessel.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A fixed bed bioreactor for culturing cells on a cell culture substrate, the fixed bed bioreactor comprising:
a cell culture vessel comprising a vessel body defining at least one interior reservoir configured to hold a multi-layered cell culture substrate, an inlet fluidly connected to the reservoir, and an outlet fluidly connected to the reservoir, the vessel body comprising a first end, a second end, and a longitudinal axis extending in a direction from the first end to the second;
a cell culture substrate disposed in the interior reservoir in a stacked arrangement; and
a guide rod disposed in the interior reservoir and extending parallel to the longitudinal axis of the vessel body,
wherein the guide rod is configured to hold in place the cell culture substrate in the interior reservoir.
wherein the cell culture substrate is a multi-layer cell culture substrate, and
wherein the guide rod is configured to maintain a rotational alignment of individual layers of the multi-layered cell culture substrate with respect to other individual layers of the multi-layered cell culture substrate.

2-5. (canceled)

6. The fixed bed bioreactor of claim 1, wherein the guide rod comprises an outer surface defining a guide rod cross-sectional profile configured to engage with the cell culture substrate.

7. The fixed bed bioreactor of claim 6, wherein the cell culture substrate comprises a cutout configured to mate with or at least partially surround the guide rod cross-sectional profile.

8. The fixed bed bioreactor of claim 1, further comprising:
an inlet distribution plate disposed between the inlet and the interior reservoir; and
an outlet distribution plate disposed between the outlet and the interior reservoir.

9. The fixed bed bioreactor of claim 8, wherein the guide rode if attached to the inlet distribution plate.

10. The fixed bed bioreactor of claim 9, wherein the second end of the vessel body is on an opposite end of the interior reservoir from the inlet distribution plate,
wherein the guide rod and the inlet distribution plate are removable from the vessel body through the second end of the vessel body.

11. The fixed bed bioreactor of claim 9, wherein the guide rod is attached to the outlet distribution plate.

12. The fixed bed bioreactor of claim 9, wherein the cell culture substrate, being at least partially confined by the inlet distribution plate and the guide rod, is removable from the vessel body.

13. The fixed bed bioreactor of claim 1, wherein the cell culture substrate comprises a structurally defined surface for culturing cells thereon, the structurally defined surface defining an ordered and regular array of openings through a thickness of the cell culture substrate.

14. The fixed bed bioreactor of claim 1, wherein at least a portion of the cell culture substrate comprises a sample substrate, the sample substrate being defined by a separation boundary between the sample substrate and a remainder of the cell culture substrate, and
wherein the separation boundary is configured to separate the sample substrate from the remainder of the cell culture substrate.

15. The fixed bed bioreactor of claim 14, wherein the separation boundary comprises at least one of the following: perforations in, cuts into or through, or locally thinned portions of the cell culture substrate.

16. The fixed bed bioreactor of claim 13, wherein the structurally defined surface comprises one of more fibers.

17. The fixed bed bioreactor of claim 16, wherein the one or more fibers of the layer are woven together in an ordered arrangement.

18. The fixed bed bioreactor of claim 1, wherein the reservoir is defined by a length and a width, the length extending from a first end of the reservoir adjacent to the inlet to a second end of the reservoir adjacent to the outlet, the cell culture matrix having a width extending substantially across a width of the reservoir.

19. The fixed bed bioreactor of claim 1, wherein the fixed bed bioreactor is configured for aseptic removal of the sample portion from the cell culture vessel.

20. The fixed bed bioreactor of claim 7, wherein each individual layer of the multi-layered cell culture substrate comprises the cutout.

21. The fixed bed bioreactor of claim 7, wherein the cell culture substrate comprises a structurally defined surface for culturing cells thereon, the structurally defined surface comprising a plurality of fibers defining an ordered and regular array of openings through a thickness of the cell culture substrate,
wherein each individual layer of the multi-layered cell culture substrate comprises the cutout, and
wherein the cutout of each individual layer of the multi-layered cell culture substrate is aligned with the cutout of every other individual layer of the multi-layered cell culture substrate, and an orientation of the plurality of fibers in one or more layers of the multi-layered cell culture substrate is different from the orientation of the plurality of fibers in other layers of the multi-layered substrate.

22. The fixed bed bioreactor of claim 21, wherein the orientation of the plurality of fibers in the one or more layers of the multi-layered cell culture substrate is rotated at a predetermined angle about the longitudinal axis of the vessel body.

23. The fixed bed bioreactor of claim 22, wherein the predetermined angle is greater than 0° and less than 90°.

24. The fixed bed bioreactor of claim 18, wherein the predetermined angle is about 45°.
